(19) United States
(12) Patent Application Publication
(10) Pub. No.: US 2019/0287679 A1
(43) Pub. Date: Sep. 19, 2019

(54) MEDICAL ASSESSMENT SYSTEM AND METHOD THEREOF

(71) Applicant: Winston Center for Attention, Language & Learning, Spokane, WA (US)

(72) Inventors: Thomas Beck, Spokane, WA (US); Wendy Popa, Spokane, WA (US)

(73) Assignee: Winston Center for Attention, Language & Learning

(21) Appl. No.: 15/923,746

(22) Filed: Mar. 16, 2018

(51) Int. Cl. G16H 50/20; G16H 10/60

(52) U.S. Cl. CPC G16H 50/20; A61B 5/165; G16H 10/60

(57) ABSTRACT

A method of assessing a medical malady. The method includes receiving response data. The response data is stored as a record in a data structure. A first data subset of select patient records is created including individuals who indicated having an improved health status after being treated for a particular malady. At least one commonality or difference is identified among the personal information. A second data subset of at least one identified commonality or identified difference is created. The first data subset is analyzed with respect to the second data subset to determine whether at least one identified commonality or identified difference is relevant to the improved health status of one or more individuals. A prediction is output of at least one of the identified commonality or the identified difference as a potential treatment for the particular malady for one or more of the plurality of individuals.

### BACKGROUND

Despite hundreds of years of improvement in the ability to diagnose and treat health maladies, misdiagnosis and ineffective treatment remain a widespread concern. For the purposes of this application, the term misdiagnosis may refer to a completely errant conclusion regarding a person's health status, or an under- or over-estimation of the severity of a person's physical and/or mental condition. Further, the term ineffective treatment may refer to a recommended treatment that did not address the malady due to any number of possibilities, including, but not limited to: a misdiagnosis, insufficient for the particular severity of the malady, unique biological resistance to the treatment, etc.

Causes for such continued misdiagnosis and ineffective treatment recommendations vary widely. Some examples of the causes include: professional error, professional ineptness, laboratory error, technical and/or technician error, machine error, translation error, documentary error, lack of access to old/new/updated information, inadequate funding for research/analysis/assessment, novelty of the malady, environmental differences, etc. In other words, there are a plethora of possible reasons for a misdiagnosis or an ineffective treatment, some of which may be challenging to control or for which can be accounted.

Thus, in view of the above concerns, there is a need for better assessment capabilities in order to accurately diagnose a malady and recommend effective treatment.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The present disclosure is not limited to the particular implementations described below.

FIG. 1 illustrates a schematic of a medical assessment system according to an example embodiment.

FIG. 2 illustrates a schematic flowchart of a medical assessment system according to an example embodiment.

FIG. 3 illustrates a schematic flowchart of additional potential steps of the medical assessment system according to an example embodiment.

FIG. 4 illustrates a schematic flowchart of additional potential steps of the medical assessment system according to an example embodiment.

FIG. 5 illustrates a schematic flowchart of additional potential steps of the medical assessment system according to an example embodiment.

FIG. 6 illustrates a schematic diagram of an illustrative computing architecture that enables data processing for medical assessment system according to an example embodiment.

### DETAILED DESCRIPTION

### Overview

This disclosure is directed to a medical assessment system via which a medical malady may be assessed and for which treatment may be recommended. More specifically, a medical assessment system as described herein may receive response data to one or more questionnaires from various sources. The response data is analyzed for an individual user (e.g., a patient) and with respect to an aggregate of response data from other users. Upon analysis, the medical assessment system may predict a potential treatment for a particular targeted malady or symptom thereof. As additional data is collected, the medical assessment system may learn and update the predictive capabilities to provide more accurate analysis and recommended treatment.

In an embodiment, the medical assessment system may include hardware and software aspects. For example, the system may include a main server or group of servers that are networked together, either at a physical onsite location or a virtual location of a cloud service, to provide and execute access and analysis functions. The server(s) may be in communication with one or more access terminals from which administrative tasks may be performed, such as maintaining and updating hardware and/or software of the system and data integrity. The system may further include remote access, user interface terminals or software modules, which communicate with the server(s) via a network connection. The network connection to access the medical assessment features of the system may include an internal, closed network accessible only to authorized facilities/terminals, a publically accessible open network via a webpage, or a combination thereof. Accordingly, users may access the user interface online and/or at a facility to enter responses to the questionnaire from any number of different locations in order to benefit from the collective data and receive recommendations for potential treatment(s) for one or more maladies. Therefore, sources of response data may include, for example, medical staff and professionals at clinics or remote sites via an electronic device capable of transmitting the data to the one or more servers, as well as other users (e.g., patients, guardians of patients, etc.) in waiting rooms at clinics, in hospitals, at home, while traveling, etc.

In the interest of protecting the privacy of patient data, while a patient's data may be associated with the patient's identity for a personal login to the user interface or for the attending medical staff and professional in order to recommend treatment for the right patient, the response data may be stripped of personal identification information with respect to the stored aggregate data. Thus, in the event of a security breach of the aggregate data stored on memory communicating with the one or more servers of the medical assessment system, personal life and health data may not be associated with any individual.

The questionnaires may be health questionnaires having a predetermined set of questions particular to patients seeking or needing treatment for one or more symptoms of a targeted medical malady. For example, for a targeted medical malady, questions may elicit responses regarding: the expression of and/or level of severity/duration/frequency of known symptoms and/or characteristics associated with the targeted medical malady; targeted personal health/diet characteristics; and patient experience with implemented previously recommended treatment(s).

Additionally, and/or alternatively, the questionnaires may be general questionnaires regarding multiple aspects (e.g., characteristics, traits, habits, etc.) of a patient's life, including, but not limited to: stress level (e.g., patient self-assessment, medical professional assessment, situational triggers, etc.); activity level (e.g., exercise habits, intensity, duration of activity or inactivity, work or school related physical activities, type of activity, etc.); sleep quality (e.g., insomnia, snoring, REM frequency and duration, etc.); personal nutrition (e.g., fats, sugars, vitamins, type and quality of foods/liquids consumed, frequency of eating, quantity of food consumed, timing of eating, etc.); study abilities (e.g., capacity to concentrate for particular lengths of time, easily distracted, etc.); genetics (e.g., family health history, known genetic defects at birth, carrier of known potentially health-adverse genes); lifestyle (e.g., wealth status relative to others in area, education status, occupation status, religion, life philosophy, hobbies, consumption of alcohol/tobacco/drugs, frequency/type/duration of exposure to media including music, pictures, video, literature, and games, etc.); physical behavior (e.g., unintended or uncontrolled movements, reactions to stimuli, abnormal actions, repetitious movements, unexpected actions, mobility characteristics, passive or aggressive motions, etc.); verbal behavior (e.g., unintended or uncontrolled vocalizations, vocalized reactions to stimuli, voice modulation, volume control, speech mannerisms, clarity, quality, etc.); home environment (e.g., urban or rural, living arrangements, care arrangements, time spent alone, responsibilities, family support, family structure, family size, level/quality of discipline, order among siblings, exposure to abuse or toxic substances, pets, amenities available, neighborhood social factors, etc.); social environment (e.g., self-imposed or situationally-imposed, level of interactivity with others from school, church, sports, work, inside home, outside home, community, etc.); geographical environment (e.g., climate, pollen exposure, altitude, average sun exposure, mold exposure, bacteria exposure, water quality, air quality, etc.); cultural environment (e.g., familial traditions, community traditions, norms, expectations, health/body treatment behavior, etc.); emotional, mental, or physical characteristics/manifestations, etc. The questionnaire may include a series of detailed questions with respect to each of the example aspects listed above. Such detailed questions may provide greater ability for the medical assessment system to predict a potential treatment that is especially tailored to benefit the individual patient based on the answers provided and a health/medical professional's review. Furthermore, the questionnaire(s) may be updated occasionally or according to a predetermined schedule to improve the predictive capability of the medical assessment system in determining a potential treatment for a malady experienced by a patient.

The response data may be input by a user, such as a patient, patient's guardian, or a medical professional, via one or more electronic sources capable of providing the response data to one or more servers of a medical assessment system, according to an embodiment. The response data may be stored in memory associated with the one or more servers to be aggregated with response data from multiple entities and analyzed individually and collectively. The analysis of the response data may be performed with respect to a particular malady according to the desire of a medical professional or a user, such as a patient, or a patient's guardian. Additionally, and/or alternatively, the response data may be analyzed broadly to determine if the data indicates any known medical malady of any kind.

As an example embodiment, a medical assessment system, such as described above, may be used to assess the patient for Attention-Deficit Disorder (ADD) and/or Attention-Deficit Hyperactivity Disorder (ADHD). Note, while the inventors recognize that a distinction exists between ADD and ADHD, for the purposes of simplicity in this specification, the acronym for ADHD is used alone hereafter as an example malady being assessed. However, it is contemplated that the medical assessment system described herein may be used to assist in assessing a plurality of maladies and one or more potential treatments therefor.

In an embodiment of a medical assessment system configured to analyze a patient for ADHD, a plurality of patients (or the patient's guardians or medical professionals treating the patients) may respond to questionnaires having questions regarding multiple aspects of a patient's life. As indicated above, the questions may be more narrowly focused on aspects known to be associated with ADHD specifically, and/or the questions may be less narrow in focus and cover a greater range of information. To obtain the response data, the patient's answers to the questionnaire(s) may be input directly into the medical assessment system via an electronic user interface or may be answered on a paper questionnaire and subsequently transferred to the system. The response data may then be stored on memory in communication with the server(s).

In an embodiment, once response data is received, a simple analysis may be performed to determine preliminary results based on known characteristics of ADHD (or other malady), and a more thorough analysis may occur thereafter. Additionally, and/or alternatively, a more thorough analysis may be performed first. In a more thorough analysis, the patient's response data may be analyzed collectively with the response data from a plurality of patients, whose answers to questionnaires were previously entered, to determine similarities and/or differences between the response data, and thus the lives, of respective patients.

In an embodiment, for analysis purposes, the answer(s) to one or more questions in the questionnaire may be weighted differently depending on the question and according to the particular malady, such as ADHD, and/or by choice of a user requesting the analysis, such as a medical professional. That is, in a situation where the user is using the medical assessment system to predict a potential treatment for a patient with ADHD for example, a question regarding a specific characteristic of that patient may be considered more relevant or important for ADHD than the same question might be considered if the patient had been being assessed for cancer. In addition to different maladies, other factors may be considered when determining how to weight different data. Examples of other factors that may affect weight given to responses in the analysis may include aspects of a patient derived from the response data itself and/or otherwise known about the user, including: age, gender, allergies, concurrent or past health situations (e.g., the patient suffers from a different condition which could prevent the implementation of a predicted potential treatment), accessibility of a predicted treatment, etc.

By analyzing the response data of a particular patient individually and compared with other patients, the medical assessment system may predict a potential treatment for a particular malady, such as ADHD. Potential treatments for a malady may be recommended based, at least in part, on a patient's personal response data, as well as similarities and/or differences in the response data as compared with respect to one or more other patients for whom a treatment appears to be or have been beneficial to the health of the one or more other patients. In an embodiment, whether a treatment is “beneficial” may be determined by an improved health status after implementation of the treatment. Thus, the questionnaire may include questions regarding a past treatment a patient has implemented and the health status of the patient after implementing the treatment, including whether the treatment was determined to be effective to treat a targeted symptom of the malady or whether the treatment was discovered to have affected the patient in an unexpected positive and/or adverse manner.

The analysis and treatment recommendation aspects of a medical assessment system may improve with the addition of response data from different individuals. That is, as additional data is received which describes the lives of a variety of people in different places, the system may learn new information that improves the predictive capability based on the diversity of the individuals and the ability to discover what commonalities are shared by those for whom treatment is effective.

### Illustrative Embodiments of a Medical Assessment System

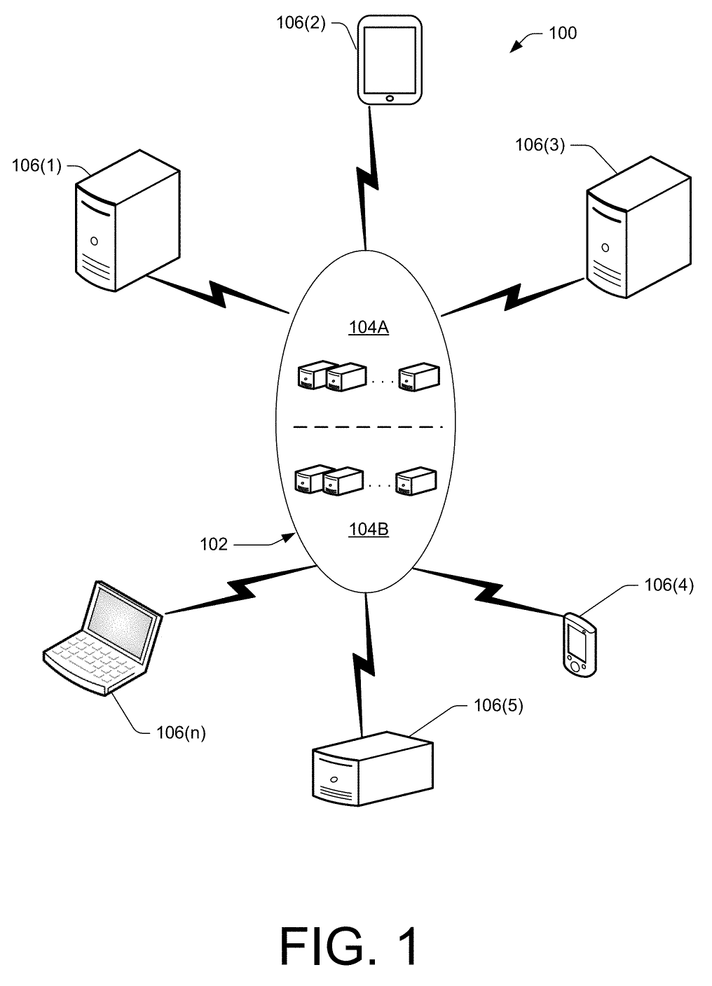

Specifically, FIG. 1 schematically illustrates a non-limiting embodiment of a medical assessment system 100 according to this disclosure. The medical assessment system 100 may include one or more servers 102 located in a centralized facility, or the one or more servers 102 may be dispersed in a plurality of locations such as a group 104A of one or more servers and a group 104B of a different set of one or more servers. Furthermore, the one or more servers 102 may be accessed via a cloud service. Regardless, in the event that multiple servers 102 are part of the system, whether the servers 102 are located together or dispersed in different regions, the servers 102 are configured to communicate with each other so as to share information therebetween.

The medical assessment system 100 further includes a user interface via which users are able to enter data in response to a questionnaire. The user interface may be accessed via an electronic device 106(1), 106(2), . . . 106(n) capable of communicating with the servers 102. The form of communication may be either directly or indirectly, where for example, a device may be able to receive response data, save the data on a memory of the device, and then subsequently the data may be transferred to a second device in communication with the servers 102. Compatible electronic devices may include, but are not limited to: desktop computers 106(1), 106(3); tablet devices 106(2); smartphones 106(4); other servers 106(5); or laptops or any other suitable electronic device 106(n).

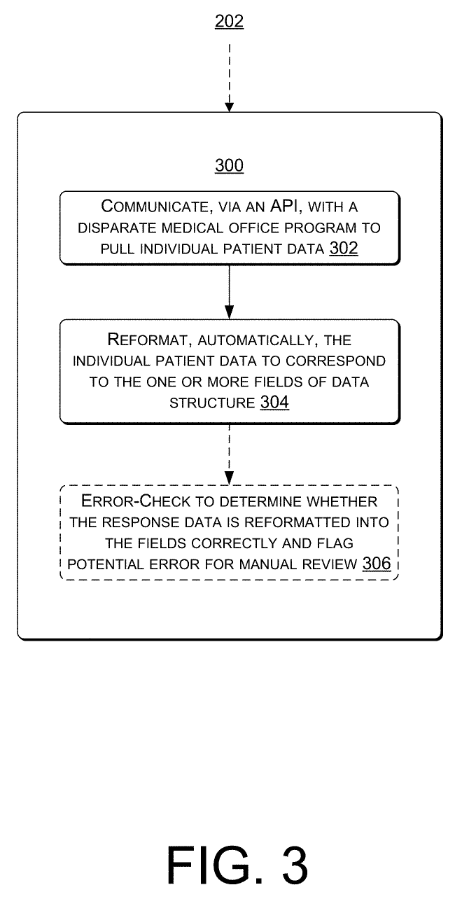

In an embodiment, the medical assessment system 100 may be implemented in a medical organization composed of multiple clinics or medical offices performing services under a single brand. Each clinic or office of the organization may provide full access to the medical assessment system 100 as an advantage to the organization's healthcare offerings. Additionally, and/or alternatively, where an office or clinic is disparate (“disparate office”) from the medical organization, the disparate office may still pay for, or otherwise have, access to the medical assessment system 100 via a network connection and the user interface. In some instances, the healthcare professionals of a disparate office may desire access to the medical assessment system 100 for one or more patients of the disparate office. However, in the event that the patients of the disparate office have not previously responded to the questionnaire of the medical assessment system 100 (possibly in another office or personally online, for example), the task of having to input all of the answers for each patient may seem daunting and tedious such that the disparate office is deterred from using the system 100. Accordingly, in an embodiment, the medical assessment system 100 may further include an application program interface (“API”) (see FIG. 3) that directly communicates with a disparate medical office program external to the medical assessment system 100. The API may communicate, for example, with a server 106(5) of the disparate office to copy individual patient data for the one or more patients. Inasmuch as the data in the disparate office is not likely to correspond directly to the questions of the questionnaire, the API may be configured to scan the data files in the disparate office program to search for information in entries that correspond to the answers of the questionnaire. Furthermore, the API may be further configured to reformat the data copied from the server 106(5) upon finding answers that correspond to the questions in the questionnaire. Thus, the medical assessment system 100 may minimize manual reentry of individual patient data that was previously stored in another data system.

In a case where response data has been copied from another data system, the medical assessment system 100 may 1) provide user access to the copied data so that a user may update, correct, or add to any incomplete or inaccurate information; and 2) flag, for review by a user, any information that appears questionable after being reformatted.

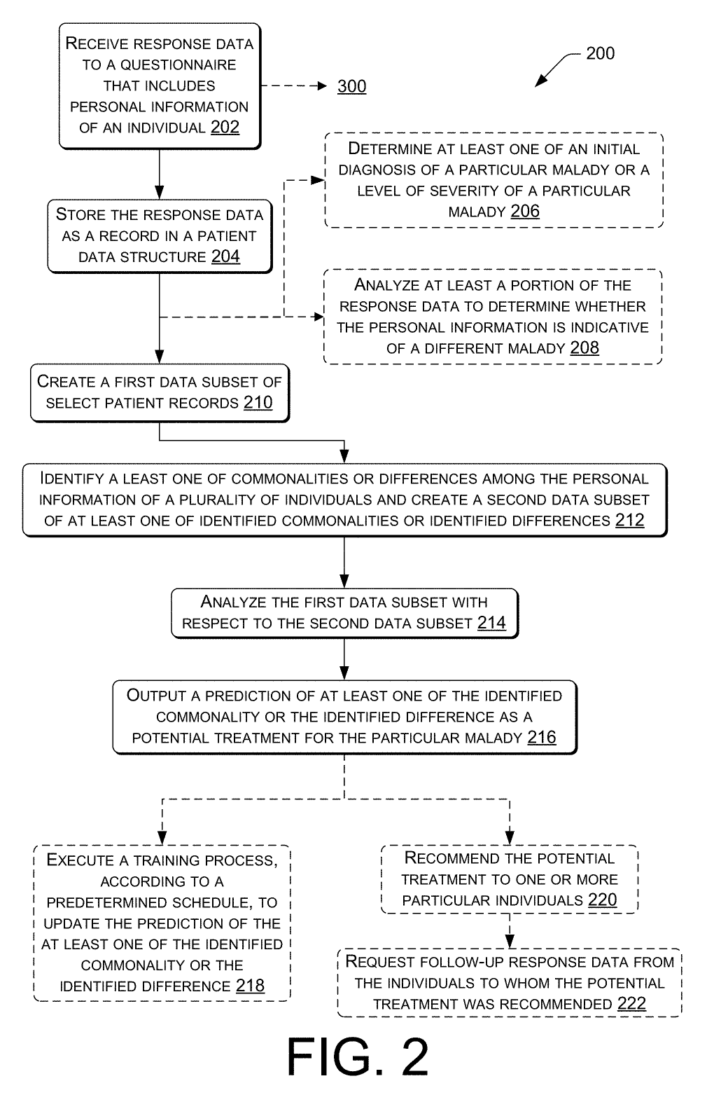

A schematic flowchart of acts of a method 200 that may be performed by a medical assessment system is depicted in FIG. 2. Note, the term “acts” as used herein and in the claims may also be referred to as steps, sub steps, or operations, interchangeably, and therefore is not a limitation on the medical assessment system 100.

Method 200 may include a step 202 in which the medical assessment system receives response data to a questionnaire that includes personal information of an individual. In step 204, the response data may be stored as a record in a patient data structure having one or more fields to store the personal information. The patient data structure is configured to store multiple records associated, respectively, with a plurality of individuals.

After response data has been received in step 202, method 200 may include an optional step 206, in which the medical assessment system may determine at least one of an initial diagnosis of a particular malady or a level of severity of a particular malady. Moreover, in optional step 208, method 200 may analyze at least a portion of the response data to determine whether the personal information is indicative of a different malady. For example, the response data may be compared with characteristics of one or more known maladies to determine whether the patient may be experiencing a different malady than was previously targeted for analysis.

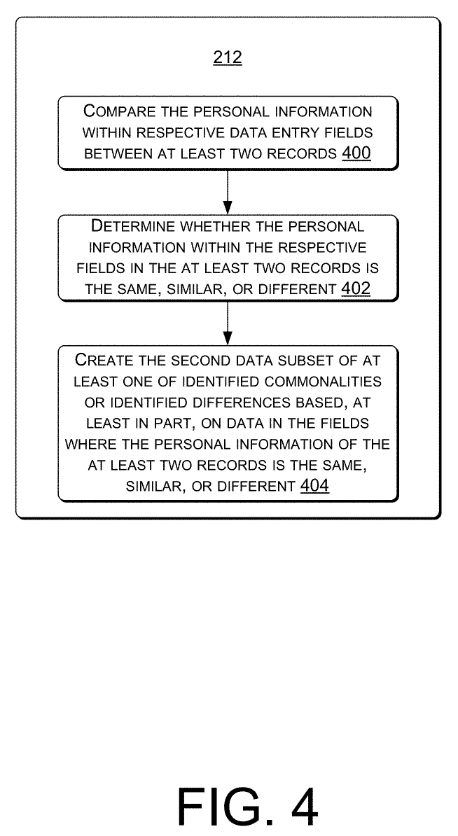

Method 200 may further include a step 210 of creating a first data subset of select patient records based, at least in part, on the response data. The select patient records may include records of one or more select individuals, of the plurality of individuals, who indicated having an improved health status after being treated for a particular malady. In step 212, at least one of commonalities or differences among the personal information of the plurality of individuals may be identified. The identification step 212 may further include substeps 400, 402, and 404 (see FIG. 4). Substep 400 compares the personal information within respective fields of the one or more fields between at least two of the records. In substep 402, a determination may be made regarding whether the personal information within the respective fields of the one or more fields in the at least two of the records is the same, similar, or different. In substep 404, a second data subset may be created including at least one of identified commonalities or identified differences based, at least in part, on data in the one or more fields where the personal information of the at least two records is determined to be the same, similar, or different.

After creation of the second data subset in step 212, method 200 may further proceed with step 214, in which the first data subset is analyzed with respect to the second data subset. The analysis may determine whether at least one of an identified commonality or an identified difference is relevant to the improved health status of the one or more select individuals. In step 216, a prediction may be output, in response to a determination that at least one of the identified commonality or the identified difference is relevant to the improved health status of the one or more select individuals, of the at least one of the identified commonality or the identified difference as a potential treatment for the particular malady.

In an embodiment, method 200 may further include optional steps 218 and 220. In step 218, a training process may be executed, according to a predetermined schedule, to update the prediction of the at least one of the identified commonality or the identified difference as a potential treatment for the particular malady. In step 220, the system may recommend the potential treatment to one or more particular individuals. In the event that a potential treatment is recommended, method 200 may further include a subsequent step 222, in which the system requests follow-up response data from any individuals to whom the potential treatment was recommended in step 220.

As discussed above, response data may be received from a variety of sources. In the event that a disparate office desires to input patient data, step 202 of method 200 may include further acts in method 300, depicted in a flowchart in FIG. 3. As depicted, in receiving response data from a disparate office, a step 302 may be performed, in which an API (discussed above) integrated in the medical assessment system 100 may communicate with a disparate medical office program to pull individual patient data. Method 300 may continue in step 304 by reformatting, automatically, the individual patient data to correspond to the one or more fields of the patient data structure of the medical assessment system 100. Furthermore, as mentioned above, an optional step 306 may be performed. Step 306 provides an error-check to determine whether the response data is reformatted into the fields correctly and may flag potential errors for manual review.

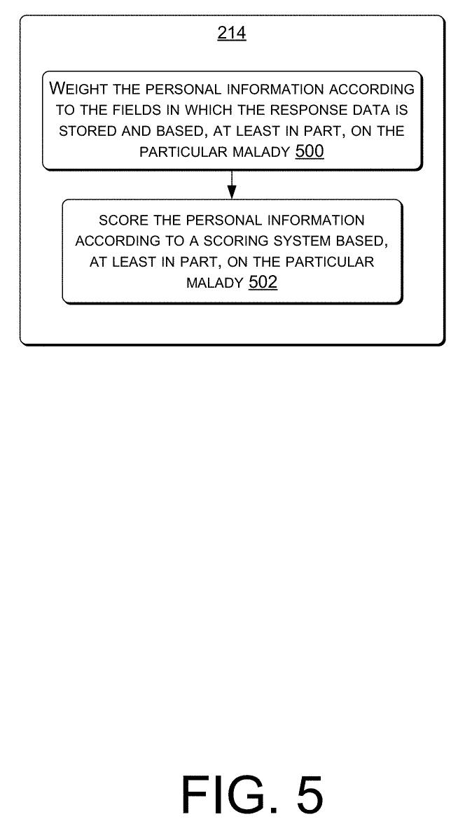

With respect to step 214 discussed above regarding the analysis of the first data subset and the second data subset, step 214 may include substep 500 and 502, as shown in FIG. 5. For example, in substep 500, the analysis process may include weighting the personal information of the response data according to which of the one or more fields in which the response data is stored. The weighting may be based, at least in part, on the particular malady of concern. Then, in substep 502, the personal information may be stored according to a scoring system, which may also be based, at least in part, on the particular malady. Inasmuch as there are many known ways to weight and score data, details of the weighting and scoring are not provided herein. Nevertheless, one skilled in the art of weighting data will understand the significance of the weighting and scoring substeps, and would know how such could be implemented.

As an example of how the medical assessment system may function, a scenario is presented in which Patient A, believed to be experiencing ADHD, submits response data to the questionnaire, which data is entered into the medical assessment system. Upon analyzing Patient A's response data in comparison with the response data of other patients, it is determined that Patient A has many life aspects in common with Patient B. Assuming Patient B has been diagnosed and treated for ADHD, and that the recommended treatment was effective to address one or more symptoms of the ADHD, the medical assessment system may return a prediction that Patient A may benefit similarly from the same treatment implemented by Patient B. Additionally, and/or alternatively, if Patient A has already tried the same treatment as Patient B, and the treatment was unsuccessful, the medical assessment system may consider the differences in the lives of the two patients, and may predict that another aspect of Patient A's life or Patient B's life may be either the cause for why the treatment was not effective for Patient A, or may be a life aspect that Patient A could implement as a potential treatment. A more specific example of the above follows:

Patients A and B are similar in age and size, and live in similar neighborhoods with similar family structures. Further, Patients A and B have, in the past, both been prescribed the same medication to address symptoms of the ADHD. The medication was effective for Patient B, but not Patient A. Analysis of the response data identified that at least one difference between the patients is Patient B's parents encourage vigorous exercise three times a week, while Patient A is generally sedentary. Thus, the medical assessment system may predict that adding exercise to Patient A's routine is a potential treatment.

Further, the medical assessment system may look at similarities between multiple patients that have had success with the same or different treatments for the same malady. The medical assessment system may determine during the analysis that the patients for whom treatment was effective are otherwise significantly different in most life aspects from each other. However, despite the numerous differences, the medical assessment system may identify one or more commonalities, and may predict that the commonalities could be potential treatments for other patients experiencing similar symptoms of a malady without successful treatments. For example, assume a group of patients of various races, ages, and lifestyles all have very few life aspects in common. However, the medical assessment system identifies that the patients of the group all happen to live within a 25 mile radius of each other in an area known to have excellent air quality. In contrast, a patient who has had no success from other treatments and who has no more life aspects in common with the group of patients than they do among each other in the group, happens to live in an area known to have very poor air quality. In such a case, the medical assessment system may predict as a potential treatment the commonality of the group, that of living where there is better air quality.

Accordingly, it is evident that a large number of potential life aspects may be analyzed and the predictions of treatment may vary widely from commonalities to differences in the response data when compared to one or many others in the system. However, by implementing the aforementioned medical assessment system to assist healthcare professionals, the medical assessment system can analyze far more data, far more quickly, efficiently, and even cost-effectively than a human could do, and recommend potential treatment that may be otherwise imperceptible, unexpected, and/or previously unknown.

### Illustrative Example of a Computing Architecture for the Medical Assessment System

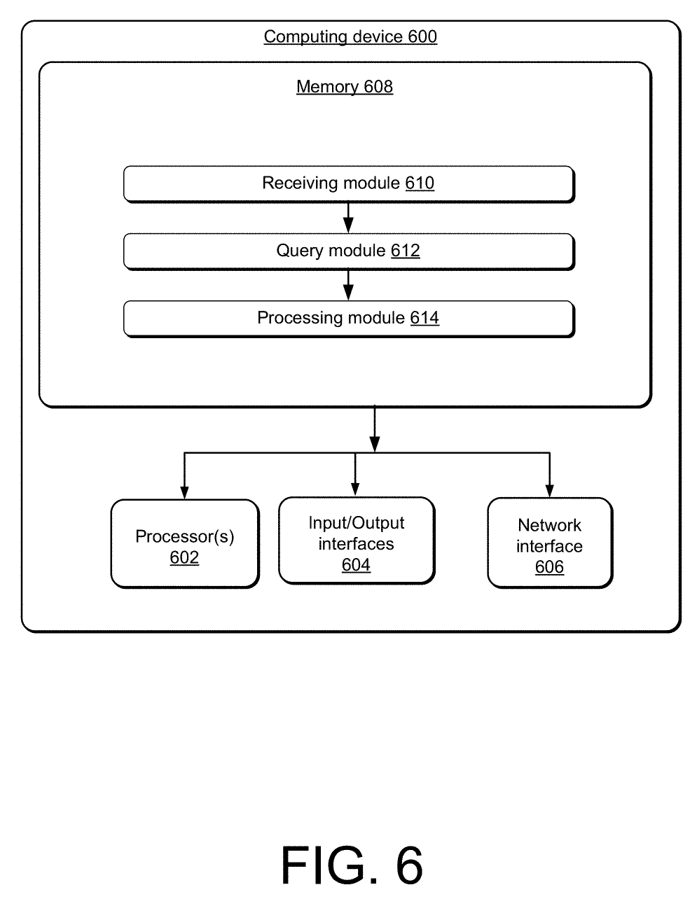

FIG. 6 is a schematic diagram of an illustrative computing architecture 600 that enables receipt and processing of the response data, as well as the analysis and predictive capabilities. The computing device 600 may be a user device via which the user accesses the user interface to input response data, or a server for processing the response data received. In an exemplary configuration, the computing device 600 may include one or more processors 602, input/output interfaces 604, a network interface 606, and memory 608.

The memory 608 may include computer-readable media in the form of volatile memory, such as random-access memory (RAM) and/or non-volatile memory, such as read only memory (ROM) or flash RAM. The memory 608 is an example of computer-readable media.

Computer-readable media includes volatile and non-volatile, removable and non-removable media implemented in any method or technology for storage of information such as computer readable instructions, data structures, program modules, or other data. Examples of computer storage media include, but are not limited to, phase change memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. As defined herein, computer-readable media does not include transitory media such as modulated data signals and carrier waves.

Turning to the memory 608 in more detail, the memory 608 may include a receiving module 610, a query module 612, and a processing module 614. The receiving module 610 may be configured to receive response data to be processed. The query module 612 may be configured to provide the questionnaire to the user(s). The processing module 614 may be configured to, in response to the receiving module 610 receiving response data as entered by a user or the API according to the questionnaire, process the response data according to method 200. Moreover, on the server side, memory 608 is configured to store the response data in a patient data structure, as previously mentioned. However, on the user device side, memory 608 may store the response data if the user desires. The response data may be stored in fields corresponding to questions in the questionnaire.

Some advantages of the medical assessment system described herein may include, for example: quicker and more accurate diagnosis and treatment of medical maladies; more efficient data collection and storage; ability to improve the system to be more accurate as additional data is gathered, etc. In essence, the organizational structure and analysis capabilities provides a potentially more effective way than could be achieved by the human brain to predict what treatments may be better for individual patients based on rapid analysis of many facts about a patient compared to similar or dissimilar facts about a large number of other people with similar conditions.

### Example Illustration of Providing Feedback to the Medical Assessment System

It is contemplated further that a health-status tracking device (not shown) may be worn by, or potentially ingested or implanted into, patients who have submitted or may submit response data to the medical assessment system. Various types of wearable devices are contemplated including devices that are embedded or placed in or on socks, shoes, wrists, ankles, backpacks, or other items worn or carried by a patient. Such devices may be network enabled to provide constant or periodic biometric and/or environmental feedback, detected from the wearer of the device and the environment, to the medical assessment system. Such feedback further enhances the ability of the medical assessment system to learn, adapt, and better predict what treatments may be more effective for individual patients.

Furthermore, based on the feedback, the medical assessment system may send recommendation communications to the health professional or directly to the user regarding predicted changes to the potential treatment.

### Conclusion

Although several embodiments have been described in language specific to structural features and/or methodological acts, it is to be understood that the claims are not necessarily limited to the specific features or acts described. Rather, the specific features and acts are disclosed as illustrative forms of implementing the claimed subject matter.

What is claimed is:

1. A medical malady treatment assessment system, comprising:
one or more processors; and
memory having instructions stored thereon, which when executed, cause the one or more processors to perform acts including:
receiving response data to a questionnaire, the response data including personal information about an individual,
storing the response data as a record in a patient data structure having one or more fields to store the personal information, the patient data structure being configured to store multiple records associated, respectively, with a plurality of individuals,
creating a first data subset of select patient records based, at least in part, on the response data, the select patient records including records of one or more select individuals, of the plurality of individuals, who indicated having an improved health status after being treated for a particular malady,
identifying commonalities among the personal information of the plurality of individuals, the identifying including:
comparing the personal information within respective fields of the one or more fields between at least two of the records,
determining whether the personal information within the respective fields of the one or more fields in the at least two of the records is the same or similar, and
creating a second data subset of identified commonalities based, at least in part, on data in the one or more fields where the personal information of the at least two records is determined to be the same or similar,
analyzing the first data subset with respect to the second data subset to determine whether an identified commonality is relevant to the improved health status of the one or more select individuals, and
outputting a prediction, in response to a determination that the identified commonality is relevant to the improved health status of the one or more select individuals, of the identified commonality as a potential treatment for the particular malady.

2. The medical malady treatment assessment system according to claim 1, wherein the personal information about which the individual is asked via the questionnaire includes environmental factors,
where environmental factors relate to a situation in which the individual lives, including at least one of a geographical environment, a residential environment, a familial structure environment, a climatological environment, an educational environment, a philosophical environment, or an occupational environment.

3. The medical malady treatment assessment system according to claim 1, wherein the personal information about which the individual is asked via the questionnaire includes personal characteristics,
where personal factors of the individual include at least one of medical information, biophysical characteristics, mental characteristics, symptomatic characteristics, genetic characteristics, or habitual characteristics.

4. The medical malady treatment assessment system according to claim 1, wherein the acts further include analyzing at least a portion of the response data to determine whether the personal information is indicative of a different malady.

5. The medical malady treatment assessment system according to claim 1, further comprising an application program interface (API) that directly communicates with a disparate medical office program external to the system,
wherein the act of receiving the response data includes:
communicating, via the API, to pull individual patient data from the disparate medical office program, and
reformatting, automatically, the individual patient data to correspond to the one or more fields of the patient data structure, thereby minimizing manual reentry of the individual patient data.

6. The medical malady treatment assessment system according to claim 5, wherein the acts further include error-checking, after the act of receiving the response data, to determine whether the response data is reformatted into the one or more fields correctly.

7. The medical malady treatment assessment system according to claim 1, further comprising:
one or more servers hosting the one or more processors and memory; and
a graphical user interface (GUI), accessible via a network connection, to provide a user access to transmit data for analysis to the one or more servers.

8. The medical malady treatment assessment system according to claim 1, wherein the acts further include executing a training process, according to a predetermined schedule, to update the prediction of the identified commonality.

9. The medical malady treatment assessment system according to claim 1, wherein the act of analyzing includes:
weighting the personal information of the response data according to which of the one or more fields in which the response data is stored and based, at least in part, on the particular malady, and
scoring the personal information according to a scoring system based, at least in part, on the particular malady.

10. The medical malady treatment assessment system according to claim 1, wherein a level of user access to the system depends, at least in part, on an amount of contribution of patient response data by the user.

11. The medical malady treatment assessment system according to claim 1, wherein the act of outputting a prediction includes processing a result of the analyzing according to a decision rule tree to assess validity of at least one of a diagnosis, a level of severity of the diagnosis, or the identified commonality as a potential treatment for the particular malady.

12. A medical malady treatment assessment system, comprising:
one or more processors; and
memory having instructions stored thereon, which when executed, cause the one or more processors to perform acts including:
receiving response data to a questionnaire, the response data including personal information about an individual,
storing the response data as a record in a patient data structure having one or more fields to store the personal information, the patient data structure being configured to store multiple records associated, respectively, with a plurality of individuals,
creating a first data subset of select patient records based, at least in part, on the response data, the select patient records including records of one or more select individuals, of the plurality of individuals, who indicated having an improved health status after being treated for a particular malady,
identifying differences among the personal information of the plurality of individuals, the identifying including:
comparing the personal information within respective fields of the one or more fields between at least two of the records,
determining whether the personal information within the respective fields of the one or more fields in the at least two of the records is different, and
creating a second data subset of identified differences based, at least in part, on data in the one or more fields where the personal information of the at least two records is determined to be different, analyzing the first data subset with respect to the second data subset to
determine whether an identified difference is relevant to the improved health status of the one or more select individuals, and
outputting a prediction, in response to a determination that the identified difference is relevant to the improved health status of the one or more select individuals, of the identified difference as a potential treatment for the particular malady.

13. The medical malady treatment assessment system according to claim 12, wherein the personal information about which the individual is asked via the questionnaire includes environmental factors,
where environmental factors relate to a situation in which the individual lives, including at least one of a geographical environment, a residential environment, a familial structure environment, a climatological environment, an educational environment, a philosophical environment, or an occupational environment.

14. The medical malady treatment assessment system according to claim 12, wherein the personal information about which the individual is asked via the questionnaire includes personal characteristics,
where personal factors of the individual include at least one of medical information, biophysical characteristics, mental characteristics, symptomatic characteristics, genetic characteristics, or habitual characteristics.

15. The medical malady treatment assessment system according to claim 12, wherein the acts further include determining at least one of an initial diagnosis of the particular malady or a level of severity of the particular malady.

16. The medical malady treatment assessment system according to claim 12, wherein the acts further include, after the act of outputting the prediction:
recommending the potential treatment to one or more particular individuals, and
requesting follow-up response data from the one or more particular individuals to whom the potential treatment was recommended.

17. A method of assessing a medical malady, the method comprising:
receiving response data to a questionnaire, the response data including personal information about an individual;
storing the response data as a record in a patient data structure having one or more fields to store the personal information, the patient data structure being configured to store multiple records associated, respectively, with a plurality of individuals;
creating a first data subset of select patient records based, at least in part, on the response data, the select patient records including records of one or more select individuals, of the plurality of individuals, who indicated having an improved health status after being treated for a particular malady;
identifying at least one of commonalities or differences among the personal information of the plurality of individuals, the identifying including:
comparing the personal information within respective fields of the one or more fields between at least two of the records,
determining whether the personal information within the respective fields of the one or more fields in the at least two of the records is the same, similar, or different, and
creating a second data subset of at least one of identified commonalities or identified differences based, at least in part, on data in the one or more fields where the personal information of the at least two records is determined to be the same, similar, or different;
analyzing the first data subset with respect to the second data subset to determine whether at least one of an identified commonality or an identified difference is relevant to the improved health status of the one or more select individuals; and
outputting a prediction, in response to a determination that at least one of the identified commonality or the identified difference is relevant to the improved health status of the one or more select individuals, of the at least one of the identified commonality or the identified difference as a potential treatment for the particular malady.

18. The method of assessing a medical malady according to claim 17, wherein the personal information about which the individual is asked via the questionnaire includes environmental factors,
where environmental factors relate to a situation in which the individual lives, including at least one of a geographical environment, a residential environment, a familial structure environment, a climatological environment, an educational environment, a philosophical environment, or an occupational environment.

19. The method of assessing a medical malady according to claim 17, wherein the personal information about which the individual is asked via the questionnaire includes personal characteristics,
where personal factors of the individual include at least one of medical information, biophysical characteristics, mental characteristics, symptomatic characteristics, genetic characteristics, or habitual characteristics.

20. The method of assessing a medical malady according to claim 17, further comprising:
weighting the personal information of the response data according to which of the one or more fields in which the response data is stored and based, at least in part, on particular malady; and
scoring the personal information according to a scoring system based, at least in part, on particular malady.
